(19) United States
(12) Patent Application Publication
(10) Pub. No.: US 2013/0281905 A1
(43) Pub. Date: Oct. 24, 2013

(54) SUPPORT-FREE SURGICAL INTERFACE FOR WOUND

(71) Applicant: Plasto Technologies SAS, Chenove (FR)

(72) Inventors: Maud STAEGER WILLIAMS, Dijon (FR); Alain GUILLEMET, Fontaine-Les-Dijon (FR)

(21) Appl. No.: 13/742,884

(22) Filed: Jan. 16, 2013

Related U.S. Application Data
(63) Continuation of application No. 12/564,347, filed on Sep. 22, 2009, now abandoned.
(60) Provisional application No. 61/101,724, filed on Oct. 1, 2008.

(30) Foreign Application Priority Data
Sep. 24, 2008 (FR) 0805247

(51) Int. Cl. A61F 13/00

(52) U.S. Cl. CPC A61F 13/00017; A61F 13/00995
USPC 602/47; 427/2.31

(57) ABSTRACT

A surgical interface for a wound, obtained from a composition including a hydrophobic matrix comprising A parts of a triblock elastomer, B parts of a plasticiser, C parts of polyeolefin, and optionally a hydrocolloid dispersed in a proportion containing 0 to 30% of the total weight of the hydrophobic matrix, with the following relations:

A=100
300<B<500
10<C<50
9%<C/(A+C)<33.5%
1.5%<C/(A+B+C)<9%
83%>B/(A+B)>75%
in which the interface composition is arranged in a thin layer on a protective support, distributed with through-holes produced in the interface arranged on the protective support, in which the interface is removed from its protective support in order to be used, and has sufficient cohesion for application to a wound.

### CROSS REFERENCE TO RELATED APPLICATION

This patent application is a continuation of U.S. application Ser. No. 12/564,347, filed on Sep. 22, 2009 which claims the benefit of U.S. Provisional Patent Application No. 61/101,724, filed Oct. 1, 2008 in the name of Maud Staeger Williams and Alain Guillemet and entitled SUPPORT-FREE SURGICAL INTERFACE FOR WOUND (as amended) and French Application No. 0805247, filed Sep. 24, 2008 and titled INTERFACE CHIRURGICALE POUR PLAIE, SANS SUPPORT, the disclosures of which are incorporated by reference herein.

### BACKGROUND OF THE INVENTION

This invention relates to a non-adherent interface intended to be applied directly in contact with a wound.

The known interfaces on the market are interfaces including a specific composition arranged on a support. Document EP 1 143 895 describes the general prior art of the invention to which reference is made, and a sterile non-adherent interface, intended to be applied directly in contact with a wound, including a support made of flexible open-mesh fabric in which the yarns are coated with a specific hydrophobic matrix gel, in which the gel coats the yarns so as to leave the mesh essentially open. Such an interface is satisfactory to use, but the technology needed to obtain a good product is difficult to implement. Indeed, it is necessary for the coating of the yarns to be absolutely complete, because it is essential to prevent any risk of direct contact between a bare fiber of the support fabric and wound healing buds, and this coating must nevertheless be limited so as not to completely obstruct the mesh and prevent exudates from passing from the wound to the outside of the interface.

U.S. Patent application 2005/0123590 discloses a hydrophobic matrix whose components can be chosen among countless families of ingredients with very different properties, and with no specific proportions being disclosed. This matrix is always applied on a porous definitive substrate, i.e. a substrate that is not removed when the dressing is applied on the skin. Regarding the manufacturing method, as shown in paragraphs 80 to 82, the substrate is porous right from the beginning and provided with substantial apertures, and the polymeric composition is applied on the substrate in a manner that should not obstruct the apertures, which makes the fabrication process not easy.

### SUMMARY OF THE INVENTION

The objective of the invention is to overcome this problem associated with the presence of a definitive substrate or support, in particular a textile support, and therefore to propose an interface not requiring a support or similar reinforcement.

The invention comprises a surgical interface for a wound, obtained from a composition including a hydrophobic matrix comprising A parts of a triblock elastomer, B parts of a plasticiser, C parts of polyeolefin, and optionally a hydrocolloid dispersed in a proportion containing 0 to 30% of the total weight of the hydrophobic matrix, with the following relations:

A=100

300<B<500

10<C<50

9%<C/(A+C)<33.5%

1.5%<C/(A+B+C)<9%

83%>B/(A+B)>75%

in which the interface composition is arranged in a thin layer on a protective support, distributed with through-holes produced in the interface arranged on the protective support, in which the interface is removed from its protective support in order to be used, and has sufficient cohesion for application to a wound.

### BRIEF DESCRIPTION OF DRAWING FIGURES

FIG. 1A is a top view of the wound dressing according to the invention; and FIG. 1B is a cross-sectional view of the wound dressing according to the invention as depicted in FIG. 1A.

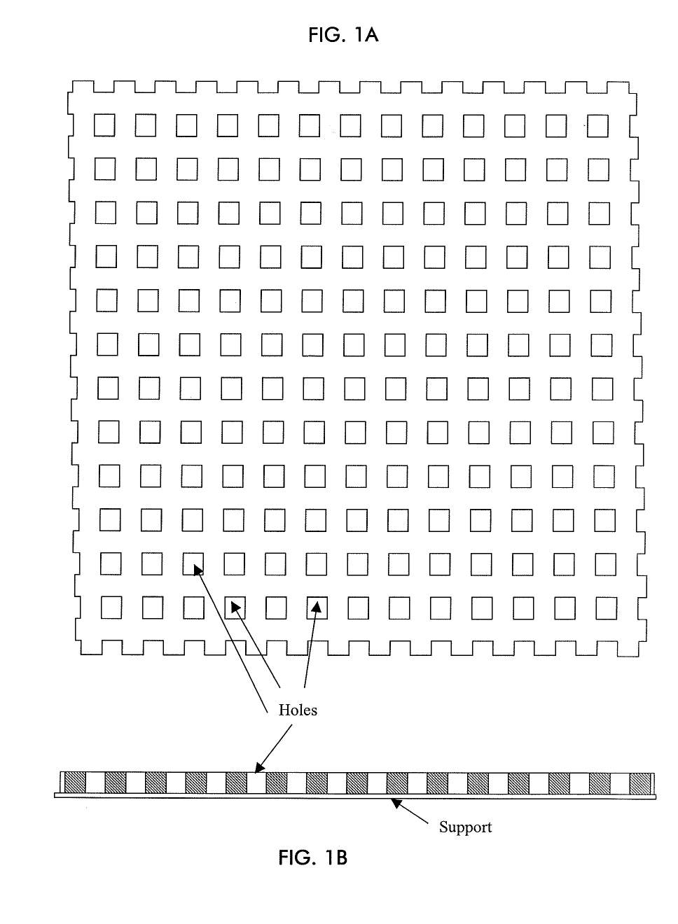

### DESCRIPTION OF EMBODIMENTS OF THE INVENTION

According to the invention, it has been discovered that it is possible to obtain a self-supporting surgical interface based on a composition including a hydrophobic matrix comprising A parts of a triblock elastomer, B parts of a plasticising agent, C parts of polyolefin, with the following relations:

A=100

300<B<500

10<C<50

9%<C/(A+C)<33.5%

1.5%<C/(A+B+C)<9%

83%>B/(A+B)>75%

wherein the interface composition is arranged in a thin layer on a temporary support, and wherein through-holes are formed in the interface.

In practice, the surgical interface is removed from its temporary support in order to be used, and the interface has sufficient cohesion for being handled and applied to a wound without any support or reinforcement.

Remarkably, the through-holes are formed in the interface during the manufacturing process at the time of it association with the temporary support, either at the very time the interface is being deposited on the temporary support or at a later step when it has been deposited. Thus according to the invention, thanks to the specific combination and proportion of ingredients, it is possible to obtain a self-supporting interface which can be used on the wound without any substrate or reinforcement (i.e. after the temporary support has been removed), which is neutral vis-à-vis the wound and its humors, and which is aerated.

Preferentially, a hydrocolloid is dispersed in the composition in a proportion ranging from 0 to 30% of the total weight of the hydrophobic matrix.

A more specific embodiment of the invention concerns a self-supporting interface based on a composition including a hydrophobic matrix comprising A parts of a triblock elastomer, B parts of a plasticising oil, C parts of polyethylene, and a hydrocolloid dispersed in a proportion containing 5 to 15% of the total weight of the hydrophobic matrix, with the following relations:

### A=100

### 300<B<500

### 10<C<50

### 9%<C/(A+C)<33.5%

### 1.5%<C/(A+B+C)<9%

### 83%>B/(A+B)>75%

The invention also concerns a manufacturing method of a self-supporting interface for wounds, comprising the steps of
preparing a composition including a hydrophobic matrix comprising A parts of a triblock elastomer, B parts of a plasticising agent, C parts of polyolefin, optionally a hydrocolloid dispersed in the composition in a proportion containing 0 to 30% of the total weight of the hydrophobic matrix, with the following relations:

### A=100

### 300<B<500

### 10<C<50

### 9%<C/(A+C)<33.5%

### 1.5%<C/(A+B+C)<9%

### 83%>B/(A+B)>75%

arranging the interface composition in a thin layer on a temporary support, and
forming through-holes in the said interface.

The through-holes are formed either simultaneously while arranging the interface on the temporary support, or after that step.

The invention also concerns the use of a surgical interface for wounds, as defined above, wherein the interface is removed from its temporary support before being used, and wherein the interface has sufficient cohesion for being handled and applied to a wound without any support or reinforcement.

Thin layer typically means an interface layer thickness between 50 μm and 2.5 mm, and preferably around 400 μm.

The triblock hydrophobic synthetic thermoplastic elastomer is advantageously an SIS-type elastomer, formed by copolymerisation advantageously of a SIS-type elastomer, formed by copolymerization of polystyrene-type blocks (for example 15%) with isoprene-type blocks. The elastomer preferably has a medium or high molecular weight and its diblock level is, for example, 18%. Such an elastomer can be plasticized with oil, as will be shown later, but the mixture can result, according to the oil ratio, in gelatinous products, soft and without self-sustenance (too high of an oil ratio), or elastic products without any adhesive property or tackiness (too low of an oil ratio).

According to the invention, the polyolefin gives to the mixture of triblock elastomer and plasticizing oil the possibility of making the final composition more rigid and capable of being handled, while being inert enough vis-à-vis the skin, and allowing the composition to be processed in a hot state. The polyolefin is preferentially polyethylene, which is provided in the form of granules. Its Brookfield viscosity is around 450 cps at 140° C. and its melting point is between 92° C. and 122° C. The amount of polyethylene with respect to the other constituents enables obtaining good rigidity without losing the tacky aspect and without whitening of the mixture. Other polyolefins, such as polypropylene, can be used in addition to or in lieu of polyethylene in the composition in order to impart rigidity to the surgical interface.

The plasticizer of the composition is preferentially oil, in particular a mineral or plant oil compatible with the other elements of the composition and tolerated by the skin. A paraffin oil with low viscosity based on paraffin and naphthenic compounds or paraffin oil mixtures are preferably used. Other plasticizers or plasticizer combinations, monomeric or polymeric, compatible with all or some of the matrix components, can be used according to the invention.

The plasticized composition is extensible and “elastic” and has a certain hysteresis with an elastic return of at least 3-5% for a 20% elongation.

To make it more anti-adherent or to maintain a moist environment suitable for healing at the interface, a hydrocolloid in dispersion is optionally added to the matrix consisting of the elements defined above. Reference can be made to the aforementioned document EP 1 143 895, which defines the nature and the conditions for addition of a hydrocolloid also suitable for the present invention. In practice, sodium carboxymethyl cellulose (NaCMC) is preferably used in proportions of 5% to 15% of the hydrophobic matrix. These particular proportions are important for limiting the percolation effect through the thickness of the product, which would entail absorption while the invention does not seek this property at all. Other hydrophilic absorbing particulate material, in reduced proportions, can also be used to help maintain a wet environment at the wound surface.

Naturally, the composition can also include various antioxidant or stabilizing products, fillers, as well as active principles that will add a specific effect to the composition. These products are known to a person skilled in the art.

The production of the interface according to the invention begins with the hot mixing without solvent according to the so-called “hotmelt” technique (at a temperature of around 150° C.) of the triblock polymer, the oily component, and the polyethylene, so as to obtain a homogeneous mixture, in which the CMC is incorporated, at a slightly lower temperature, for example between 130° C.-135° C. The product of the mixture is then deposited as a coating with a chosen thickness onto a support. The support can be a continuous or discontinuous support, and it can be a temporary support serving only during the manufacturing process, or a protective temporary support serving for transport and storage of the interface until use thereof. The support can be of any type once it has a non-adhesive surface enabling easy separation of the interface and the support. The temporary protective support is advantageously a flexible support, such as a siliconized sheet (film), for example a siliconized polyester sheet. Another siliconized sheet is added, either quasi-simultaneously or in a subsequent step, on top of the interface so that the latter is protected on both of its faces, until it is applied onto a patient's wound. Reference can be made, for a technique of coating between two supports, to the figure on page 153 of the work “Coating and Laminating”, Herbert Weiss, Converting Technology Machine, 1977.

According to an essential aspect of the invention, the process involves the formation of holes in the coated interface.

According to a first embodiment, the holes are formed by a method of perforation (for example, by punch and die) of the coating when it is already deposited on its flexible support, and preferably when it is protected by its two flexible protective sheets.

According to a second embodiment, the holes are formed in the coating at the same time that it is deposited on its support, for example by a machine sold by Cavitec under the reference Cavimel-TSM or by Nordson under the reference “REA system”, which process automatically leaves the coating in the form of a screen. The principle is to deposit, using a nozzle, the product on a screened or etched roll, which then transfers the screened coating onto its temporary protective support. Such a process is known, for example, from document WO/011352.

The holes have an average diameter of 1 to 4 mm, and, for example, are 3-mm round holes with a pitch of 8 mm.

The interface of the invention is intended to be used alone, once separated from its protective support(s) and deposited on the wound. It exhibits several features favorable for its application:
a certain cohesion and rigidity which make it possible to be handled by the caregiver staff, and it can then be removed entirely without leaving any residual parts after use;
transparency which enables the caregiver staff to monitor the evolution of the wound healing without having to remove the interface;
tackiness that enables it to remain alone in place on the patient's skin, even if the patent changes positions. This tackiness nevertheless does not imply notable adhesion in the measurable sense of the term, as the interface of the invention is specifically non-sticking and does not cause any tearing of human tissue or healing buds when it is removed.

Once the interface according to the invention has been placed on a wound, it can be combined with an additional layer, on the side opposite the wound, for example, a hydrophilic compress (gauze, polyurethane foam as sold under the trademarks Rynel® and Corpura®, or absorbent non-woven fabrics as sold by the Freudenberg company) arranged above the interface.

It is also possible to combine this additional layer with the interface when it is on its protective temporary support. The interface-additional layer complex is then placed on the wound as a unit, by placing the interface on the side of the wound, thereby maintaining the compress at a distance from the wound and preventing its fibres from interfering with the wound or the healing thereof.

Table 1 below shows the data of a first series of experiments obtained on the basis of six examples. In each of these examples, a composition was produced in the proportions indicated in the table, using the following products:

The triblock polymer used is a styrene-isoprenestyrene (SIS/SI) linear triblock/diblock copolymer of the 10 brand Vector 4113A.

The plasticiser is a white, flavourless and odourless pharmaceutical oil of the brand Ondina 917 (Shell).

The polyethylene is a homopolymer A-C 8 (Honeywell).

The NaCMC is a purified sodium carboxymethyl cellulose 15 of grade 7H4XF.

The stabilizer is a phenolic antioxidant of the brand Irganox 1010 (Ciba).

First, all of the elastomer and ⅔ of the oil were mixed at an indicated temperature of 175° C. for 60 minutes, then all of the polyethylene and the remainder of the oil were added. The heating of the mixture was then stopped, and the NaCMC was incorporated and mixed for 30 minutes.

The composition was then deposited on a support by simple coating.

Round perforations 3 mm in diameter were made using a rotary punching tool with a circumference of 508 mm and a diameter of 161 mm.

The properties of the various samples were then evaluated, by observing their resistance (the product must be neither too soft nor too rigid or brittle), their color (the product must neither be white nor lose its transparency) and their tackiness, i.e. their ability to remain by themselves on a patient's skin, for example on an arm turned over, without falling off too soon (the time before falling off is measured). These tests showed good performance of samples 3 to 6 and confirmed the value of the parameters chosen.

To confirm this data, we then tested a wider range of samples of compositions as reported in the appended table 2. The results also show that compliance with the criteria indicated herein enabled samples to be obtained with the good functional properties required in order to produce a support-free interface. In addition, we obtained Brookfield viscosity measurements “rolling ball tack” measurements (path in mm covered by a ball rolled over a tacky product according to standard ASTM D 3121) and strength measurements in cN/cm during a 20% tensile elongation cycle. All of the measurements correlate the observations made, in which the increase in the PE level reduces the tacky character of the composition and makes the product more rigid, difficult to stretch and less transparent, hence the choice of the upper limit of PE according to the invention.

Classical absorption tests made on samples according to the invention show absorbency levels below 1%, in contradistinction with the at least 50% sought by the prior art US 2005/0123590.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

TABLE 1

| Ex | 1 | 2 | 3 | 4 | 5 | 6 |
|---|---|---|---|---|---|---|
| A (SIS) | 100 | 100 | 100 | 100 | 100 | 100 |
| B (Oil) | 567 | 567 | 425 | 350 | 250 | 400 |
| C (PE) | 67 | 100 | 50 | 25 | 20 | 20 |
| CMC | | 76 | 57 | 47 | 77 | 51.5 |
| A + B + C | 734 | 767 | 575 | 475 | 370 | 520 |
| A/(A + B + C) | 13.6 | 13 | 17.4 | 21.1 | 27 | 19.2 |
| B/(A + B + C) | 77.2 | 73.9 | 73.9 | 73.7 | 67.6 | 76.9 |
| C/(A + B + C) | 9.1 | 13 | 8.7 | 5.3 | 5.4 | 3.8 |
| A/(A + B) | 15 | 15 | 19 | 22.2 | 28.6 | 20 |
| B/(A + B) | 85 | 85 | 81 | 77.8 | 71.4 | 80 |
| C/(A + C) | 40.1 | 50 | 33.3 | 20 | 16.7 | 16.7 |
| Module - | 20.2 | 18.4 | 22.6 | 21.4 | — | |
| Prop | brit. | brit. | GR | GR | brit. | GR |
| | | white | white | | | |
| | | greasy | good T | good T | good T | |

GR = Good resistance
T = Tackiness
Brit. = brittle
Prop = properties

TABLE 2

| | Series HM-D2334 | | | | | | Series HM-D2327 | | | | |
|---|---|---|---|---|---|---|---|---|---|---|---|
| Composition | A | B | C | D | E | F | A | B | C | D | E |
| A (SIS) | 100 | 100 | 100 | 100 | 100 | 100 | 100 | 100 | 100 | 100 | 100 |
| B (Oil) | 500 | 500 | 500 | 500 | 500 | 500 | 400 | 400 | 400 | 400 | 400 |
| C (PE) | 0 | 10 | 20 | 40 | 60 | 80 | 0 | 20 | 40 | 60 | 80 |
| Carboxymethyl | 59 | 60 | 61 | 63 | 65 | 67 | 49.5 | 51.5 | 53.5 | 55.4 | 57.4 |
| Cellulose | | | | | | | | | | | |
| C/(A + B + C) | 0% | 1.5% | 2.9% | 5.7% | 8.3% | 10.7% | 0% | 3.5% | 6.7% | 9.8% | 12.6% |
| C/(A + C)) | 0% | 9% | 17% | 29% | 38% | 44% | 0% | 17% | 29% | 38% | 44% |
| C/(A + B + C)) | 0% | 2% | 3% | 6% | 9% | 12% | 0% | 4% | 7% | 11% | 14% |
| B/(A + B) | 83.3% | 83.3% | 83.3% | 83.3% | 83.3% | 83.3% | 80% | 80% | 80% | 80% | 80% |
| Compliance | NO | NO | NO | NO | NO | NO | NO | YES | YES | NO | NO |
| with patent | | | | | | | | | | | |
| criteria | | | | | | | | | | | |
| Brookfield | 35 | 65 | 125 | 280 | 4815 | 5875 | 3125 | 3500 | 3750 | 4250 | 2125 |
| viscosity at | | | | | | | | | | | |
| 120° C., mod | | | | | | | | | | | |
| 27 (mPa · s) | | | | | | | | | | | |
| Tack rolling | 0 | 0 | 1093 | 903 | 1147 | 1330 | 970 | 670 | 800 | 1310 | >1500 |
| ball/PSTC- | | | | | | | | | | | |
| 6/ASTM | | | | | | | | | | | |
| D3121 (mm) | | | | | | | | | | | |
| Longitudinal | 0 | 0 | 1 | 1.5 | 6 | 8.1 | 2.1 | 4.1 | 5.1 | 6.4 | 11.1 |
| force for 20% | | | | | | | | | | | |
| elongation | | | | | | | | | | | |
| Transparency | Ok | Ok | Light | White | White | White | Ok | Ok | Ok/light | White | White |
| Resistance | Soft | Soft | Soft | Soft | Ok | Brittle | Soft | Ok | Ok | Brittle | Brittle |
| Adhesive | Greasy | Greasy | Greasy | Ok | Ok | Ok | Greasy | Ok | Ok | Dry | Dry |
| character | | | | | | | | | | | |
| Coatability | Easy | Easy | Easy | Easy | Easy | Easy | Easy | Easy | Easy | Easy | Easy |
| Compliance | | | | | | | | Good | Good | | |
| with functional | | | | | | | | functional | functional | | |
| criteria | | | | | | | | properties | properties | | |

| | Series HM-D2315′ | | | | | Series HM-D2331′ | | | | |
|---|---|---|---|---|---|---|---|---|---|---|
| Composition | A | B | C | D | E | A | B | C | D | E |
| A (SIS) | 100 | 100 | 100 | 100 | 100 | 100 | 100 | 100 | 100 | 100 |
| B (Oil) | 350 | 350 | 350 | 350 | 350 | 300 | 300 | 300 | 300 | 300 |
| C (PE) | 0 | 20 | 40 | 60 | 80 | 0 | 20 | 40 | 60 | 80 |
| Carboxymethyl | 44.6 | 46.5 | 48.5 | 50.5 | 52.5 | 39.6 | 41.6 | 43.6 | 45.5 | 47.5 |
| Cellulose | | | | | | | | | | |
| C/(A + B + C) | 0% | 3.9% | 7.4% | 10.7% | 13.7% | 0% | 4.3% | 8.3% | 11.9% | 15.17% |
| C/(A + C)) | 0% | 17% | 29% | 38% | 44% | 0% | 17% | 29% | 38% | 44% |
| C/(A + B + C)) | 0% | 4% | 8% | 12% | 15% | 0% | 5% | 9.1% | 13% | 17% |
| B/(A + B) | 77.8% | 77.8% | 77.8% | 77.8% | 77.8% | 75% | 75% | 75% | 75% | 75% |
| Compliance | NO | YES | YES | NO | NO | NO | YES | NO | NO | NO |
| with patent | | | | | | | | | | |
| criteria | | | | | | | | | | |
| Brookfield | 2750 | 4375 | 6500 | 7000 | 1375 | 8250 | 13000 | 20000 | 4500 | 1750 |
| viscosity at | | | | | | | | | | |
| 120° C., mod | | | | | | | | | | |
| 27 (mPa · s) | | | | | | | | | | |
| Tack rolling | 895 | 738 | 1033 | 1255 | >1500 | 1070 | 790 | 1440 | >1500 | >1500 |
| ball/PSTC- | | | | | | | | | | |
| 6/ASTM | | | | | | | | | | |
| D3121 (mm) | | | | | | | | | | |
| Longitudinal | 2 | 3.5 | 4.8 | 9.8 | 11.7 | 3.1 | 4.8 | 6.5 | 14.3 | 16.8 |
| force for 20% | | | | | | | | | | |
| elongation | | | | | | | | | | |
| Transparency | Ok | Ok | Light | White | White | Ok | Ok/light | White | White | White |
| Resistance | Soft | Ok | Ok | Brittle | Brittle | Soft | Ok | Brittle | Brittle | Brittle |
| Adhesive | Greasy | Ok | Ok | Dry | Dry | Greasy | Ok | Dry | Dry | Dry |
| character | | | | | | | | | | |
| Coatability | Easy | Easy | Easy | Easy | Easy | Easy | Difficult | Difficult | Difficult | Difficult |
| Compliance | | Good | Good | | | | Good | | | |
| with | | functional | functional | | | | functional | | | |
| functional | | properties | properties | | | | properties | | | |
| criteria | | | | | | | | | | |

1. A self-supporting surgical interface comprising a composition that includes a hydrophobic matrix, the hydrophobic matrix comprising A parts of a triblock elastomer, B parts of a plasticising agent, C parts of polyolefin, with the following relations:
A=100
300<B<500
10<C<50
9%<C/(A+C)<33.5%
1.5%<C/(A+B+C)<9%
83%>B/(A+B)>75%
wherein the interface composition is arranged in a thin layer on a temporary support, wherein distributed through-holes are formed in the said interface, and
wherein the interface is removed from its temporary protective support in order to be used and can be handled and applied to a wound without any additional support or reinforcement.

2. (canceled)

3. The surgical interface of claim 1, comprising a hydrocolloid dispersed in the composition in a proportion containing 0 to 30% of the total weight of a hydrophobic matrix.

4. A surgical interface for a wound, obtained from a composition including a hydrophobic matrix comprising A parts of a triblock elastomer, B parts of a plasticising oil, C parts of polyethylene, and a hydrocolloid dispersed in a proportion containing 5 to 15% of a total weight of the hydrophobic matrix, with the following relations:
A=100
300<B<500
10<C<50
9%<C/(A+C)<33.5%
1.5%<C/(A+B+C)<9%
83%>B/(A+B)>75%
in which the composition is arranged in a thin layer on a protective support, distributed with through-holes produced in the interface arranged on the protective support, in which the interface is removed from its protective support in order to be used, and can be handled and applied to a wound without any additional support or reinforcement.

5. The interface according to claim 1, wherein the triblock elastomer is a SIS-type elastomer, formed by copolymerisation of polystyrene-type blocks with isoprene-type blocks.

6. The interface according to claim 1, wherein the plasticing agent is oil.

7. The interface according to claim 3, wherein the hydrocolloid is sodium carboxymethyl cellulose (CMC).

8. The interface according to claim 1, wherein the interface is coated on the protective support.

9. The interface according to claim 8, wherein the through-holes comprise perforations of the interface that is coated on the protective support.

10. The interface according to claim 8, wherein the through-holes are formed by a screened coating.

11. The interface according to claim 1, further comprising an additional layer on a side opposite the wound.

12. A manufacturing method of a self-supporting interface for wounds, comprising the steps of
preparing an interface composition including a hydrophobic matrix comprising A parts of a triblock elastomer, B parts of a plasticising agent, C parts of a polyolefin, with the following relations:
A=100
300<B<500
10<C<50
9%<C/(A+C)<33.5%
1.5%<C/(A+B+C)<9%
83%>B/(A+B)>75%
arranging the interface composition in a thin layer on a temporary support,
forming through-holes in the interface.

13. The method of claim 12, comprising forming the through-holes either simultaneously while arranging the interface on the temporary support, or after the arranging step.

14. The interface according to claim 4, wherein the triblock elastomer is a SIS-type elastomer, formed by copolymerisation of polystyrene-type blocks with isoprene-type blocks.

15. The interface according to claim 4, wherein the hydrocolloid is sodium carboxymethyl cellulose (CMC).

16. The interface according to claim 4, wherein the interface is coated on the support.

17. The interface according to claim 16, wherein the through-holes comprise perforations of the interface that is coated on the support.

18. The interface according to claim 4, further comprising an additional layer on the side opposite the wound.

19. The method of claim 12, wherein the hydrophobic matrix, further comprises a hydrocolloid dispersed in the composition in a proportion containing 0 to 30% of total weight of the hydrophobic matrix.
